(19) United States
(12) Patent Application Publication
(10) Pub. No.: US 2012/0220903 A1
(43) Pub. Date: Aug. 30, 2012

(54) PERIODONTAL FORCE PROBE

(76) Inventors: Boris A. SIMMONDS, Hoover, AL (US); Fred J. Molz, IV, Birmingham, AL (US); J. Todd Strong, Vestavia, AL (US); Michael Reddy, (US)

(21) Appl. No.: 13/036,806

(22) Filed: Feb. 28, 2011

(51) Int. Cl. A61B 5/107

(52) U.S. Cl. 600/589

(57) ABSTRACT

An apparatus for material characterization including a probe for applying a force to a defined section of biologic tissue. The apparatus also includes a sensor to record the force applied onto the biologic tissue by the probe. And, the apparatus includes a data transmission interface to transmit the recorded force to a data processor.

### TECHNICAL FIELD

The present invention relates generally to the field of periodontal tools, and more particularly to electro-physical periodontal tools used to evaluate gingival and osseous tissues.

### BACKGROUND

The human periodontal ligament (PDL) is a group of connective tissue fibers that attaches a tooth to the alveolar bone and provides nutritive, proprioceptive, and reparative functions. In particular, the PDL is composed of collagenous fibers and a gelatinous ground substance including cells and neurovascular tissue. Biomechanically, the ligament demonstrates nonlinear viscoelasticity. Patient outcomes associated with dental treatments are strongly influenced by the mechanical support of the PDL. Therefore, testing the health of the PDL is increasingly important during routine examinations. Functional masticatory forces are relevant in prosthodonictics and periodontics, whereas low, continuous forces are typically applied in orthodontic treatments. Finite element (FE) analyses that have simulated clinical experiments of dental implants, operative dentistry, prosthodontics, and orthodontics may be limited by their use of relatively simple (linear elastic) material properties of the PDL. Studies using quasi-linear viscoelastic theory have been performed to quantify the non-linear, time-dependent PDL stress-strain behavior. An example study is described in “Quasi-Linear Viscoelastic Behavior of the Human Periodontal Ligament” (2002) by Toms, Dakin, Lemons and Eberhardt.

The interface between a tooth and the surrounding gingival tissue is a dynamic structure. The gingival tissue forms a crevice surrounding the tooth, similar to a miniature, fluid-filled moat, wherein food debris, endogenous and exogenous cells, and chemicals float. The depth of this crevice, known as a sulcus, is in a constant state of flux due to microbial invasion and subsequent immune response. Located at the depth of the sulcus is the epithelial attachment, consisting of approximately 1 mm-2 mm of junctional epithelium and another 1 mm-2 mm of gingival fiber attachment, comprising the approximately 2 mm-4 mm of biologic width naturally found in the oral cavity. The sulcus is literally the area of separation between the surrounding epithelium and the surface of the encompassed tooth.

The normal sulcular depth is three millimeters or less. Through much investigation and research, it has been determined that sulcular depths of three millimeters or less are readily self-cleansable with a properly used toothbrush or the supplemental use of other oral hygiene aids. When the sulcular depth is chronically in excess of three millimeters, regular home care is unable to properly cleanse the full depth of the sulcus, allowing food debris and microbes to accumulate. This poses a danger to the periodontal ligament (PDL) fibers that attach the gingiva to the tooth. If accumulated microbes remain undisturbed in a sulcus for an extended period of time, they will penetrate and ultimately destroy the delicate soft tissue and periodontal attachment fibers. If left untreated, this process may lead to a deepening of the sulcus, recession, destruction of the periodontium, and tooth loss.

A gingival pocket presents when the marginal gingiva experiences an edematous reaction, whether due to localized irritation and subsequent inflammation, systemic issues, or drug induced gingival hyperplasia. Regardless of the etiology, when gingival hyperplasia occurs, greater than normal (the measurement in a pre-pathological state) periodontal probing measurements can be read, creating the illusion that periodontal pockets have developed. This phenomena is also referred to as a false pocket or “pseudopocket”. The epithelial attachment does not migrate, but simply remains at the same attachment level found in health. The only anatomical landmark experiencing migration is the gingival margin in a coronal direction.

In a gingival pocket, no destruction of the connective tissue fibers (gingival fibers) or alveolar bone occurs. This early sign of disease in the mouth is completely reversible when the etiology of the edematous reaction is eliminated and frequently occurs without the need for dental surgical therapy. However, in certain situations, a gingivectomy is necessary to reduce the gingival pocket depths to a healthy 1-3 mm.

Tools used to evaluate and characterize periodontal human tissue are useful in maintaining the health of an individual. And, monitoring disease and pathology can greatly affect the mechanical properties of human tissue because if weaknesses are detected early enough, the individual can initiate corrective action. Periodontal-specific probes currently available measure pocket/sulcus depth. The more advanced of these available probes find the pocket/sulcus depth automatically using a mechanical means to ensure a semi-standard force is applied. These probes do not, however, characterize the tissue as a material continuum and instead only characterize the tissue in its geometry.

It is to the provision of meeting these and other needs that the present invention is primarily directed.

### SUMMARY

In example embodiments, the present invention provides a periodontal probe that measures periodontal health by interpreting a resistive force applied to a probe in contact with a defined region of gum tissue.

In one aspect, the present invention relates to a material characterization apparatus including a probe for applying a force to a defined section of biologic tissue. The apparatus further includes a sensor to record the force applied onto the biologic tissue by the probe and a data transmission interface to transmit the recorded force to a data processor.

In another aspect, the invention relates to a material characterization apparatus including a predictably flexible probe for applying a uni-directional force to a defined section of biologic tissue. The apparatus also includes a sensor secured to the flexible probe. And, the sensor records strain data generated by the flexible probe upon application of the uni-directional force to the biologic tissue. The apparatus further includes an interface in electronic communication with the sensor. The communication interface sends the strain data to a data processor.

In still another aspect, the invention relates to a material characterization apparatus including a force-application member that physically reacts to a resistive force generated by the surface upon which a force is applied. The apparatus also includes a sensor that records data describing the physical reaction of the force-application member. And, the apparatus includes a data transmitter that digitally transmits the data recorded by the sensor to a remote receiver.

In still another aspect, the invention relates to a system for characterizing material including a contact member for applying a force to a defined section of material and a sensor to record the force applied onto the material by the contact member. The system also includes a data communicator to communicate the recorded force and a data receiver to receive the communicated recorded force.

In still another aspect, the invention relates to a method for characterizing material including applying a force to a material with a contact member comprising predictable flexibility and recording a material resistive force with a sensor in flexible communication with respect to the contact member. The method also includes communicating the recorded resistive force through a data communicator.

In still another aspect, the invention relates to an apparatus for material characterization including a contact member for applying a force to a defined section of material and a sensor to record the force applied onto the material by the contact member. The apparatus also includes a displacement instrument secured with respect to the contact member. This displacement member recognizes the physical displacement of the contact member with respect to a fixed geometry secured within the defined section of material to be characterized.

These and other aspects, features and advantages of the invention will be understood with reference to the drawing figures and detailed description herein, and will be realized by means of the various elements and combinations particularly pointed out in the appended claims. It is to be understood that both the foregoing general description and the following brief description of the drawings and detailed description of the invention are exemplary and explanatory of preferred embodiments of the invention, and are not restrictive of the invention, as claimed.

### BRIEF DESCRIPTION OF THE DRAWINGS

FIG. 1 is a perspective view of a probe according to an example embodiment of the present invention.

FIG. 2A is a bottom plan view of the probe shown in FIG. 1 as seen looking upward from underneath.

FIG. 2B is a front elevation view of the probe shown in FIG. 1 as viewed facing toward the probe tip.

FIG. 2C is a side elevation view of the probe shown in FIG. 1 as viewed from the side.

FIG. 2D is a rear elevation view of the probe shown in FIG. 1 as viewed looking toward the base.

FIG. 3 is a perspective cross-sectional view of the probe shown in FIG. 1 having a handle.

FIG. 4 is a flow chart describing an example method of using a probe in accordance with the present invention.

FIG. 5 is a perspective view of the probe shown in FIG. 1 coupled with a deflection instrument and having a handle.

FIG. 6 is a side elevation view of the probe, displacement instrument and handle shown in FIG. 5, shown in use with respect to tissue and a dental prosthesis.

FIG. 7 is a side elevation view of the probe, displacement instrument and handle shown in FIG. 5, shown in a relaxed state and a strained state.

FIG. 8 is a perspective view of a probe according to an additional example embodiment of the invention, shown in use with a load cell.

FIG. 9 is a diagram of a probe according to another example embodiment of the present invention, shown in a relaxed state and a pivoted state contacting a load cell.

FIGS. 10A-10D are several views showing an example mount for securing a probe in accordance with the present invention.

### DETAILED DESCRIPTION OF EXAMPLE EMBODIMENTS

The present invention may be understood more readily by reference to the following detailed description of the invention taken in connection with the accompanying drawing figures, which form a part of this disclosure. It is to be understood that this invention is not limited to the specific devices, methods, conditions or parameters described and/or shown herein, and that the terminology used herein is for the purpose of describing particular embodiments by way of example only and is not intended to be limiting of the claimed invention. Any and all patents and other publications identified in this specification are incorporated by reference as though fully set forth herein.

Also, as used in the specification including the appended claims, the singular forms “a,” “an,” and “the” include the plural, and reference to a particular numerical value includes at least that particular value, unless the context clearly dictates otherwise. Ranges may be expressed herein as from “about” or “approximately” one particular value and/or to “about” or “approximately” another particular value. When such a range is expressed, another embodiment includes from the one particular value and/or to the other particular value. Similarly, when values are expressed as approximations, by use of the antecedent “about,” it will be understood that the particular value forms another embodiment.

In one example embodiment, the present invention is an apparatus for material characterization used to consistently measure pocket/sulcus depth and characterize tissue as a material continuum. The apparatus can also enable a user to determine adequate mechanical characterization of material, for example soft-tissue integration onto, and nearby, a fixed geometry, for example a tooth, dental implant or dental prosthesis. This is of particular importance because a true sulcus or cemento-enamel junction does not form with respect to a dental prosthesis. And, data obtained from the apparatus preferably allows for continual, accurate monitoring of oral tissue health by a user. Additionally, the apparatus can be used to characterize a variety of materials which have structural preferences.

In example embodiments, the apparatus characterizes gingival and osseous tissues in-vivo via electro-physical means. For example, in one embodiment, the apparatus includes a sensor that responds to stress or strain produced by probing tissue materials. In such embodiments, the apparatus determines and returns quantitative and qualitative mechanical response data to the user using a wireless device for data communication, through a data communicator, from the apparatus and a wireless receiver for data acquisition, graphic display and interpretation. Alternatively, the response data can be returned to the user using a wired device. Optionally, the apparatus can return the response data to the user in real time. And, the apparatus can provide data suitable for deriving output, for example viscoelastic mechanical response curves, energy absorbed and/or peak applied-force values with in-vivo human tissue.

Preferably, the apparatus is capable of being submerged within a sterilizing solvent without sustaining damage to electrical components. This ability ensures that all surfaces open to blood-borne pathogens can be thoroughly cleaned and/or sterilized. Alternatively and/or additionally, the apparatus can also be autoclavable to ensure that levels of cleanliness equivalent to standard periodontal probing tools are maintained and the necessity for disposable systems attached directly to a probe handle tip is reduced.

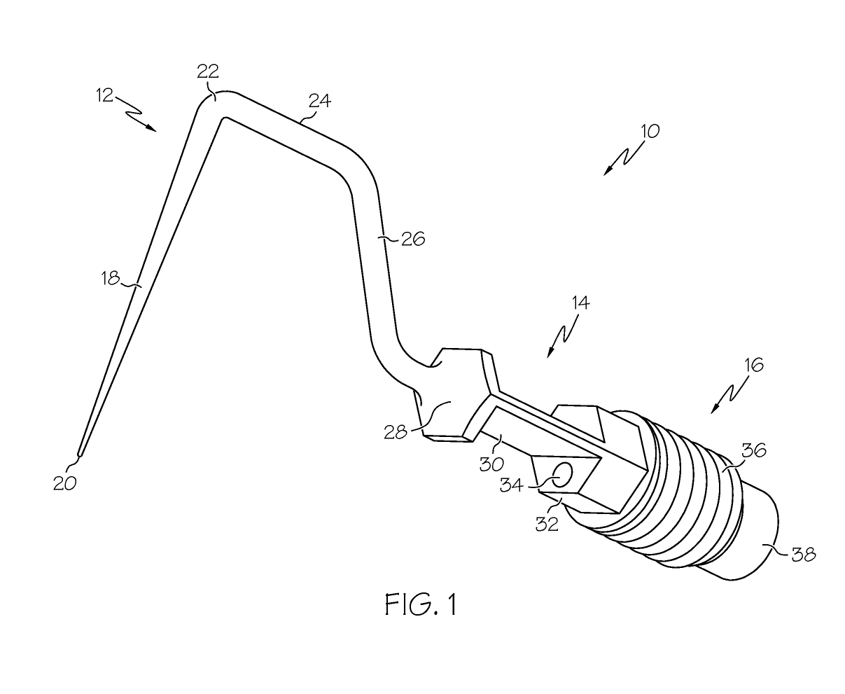

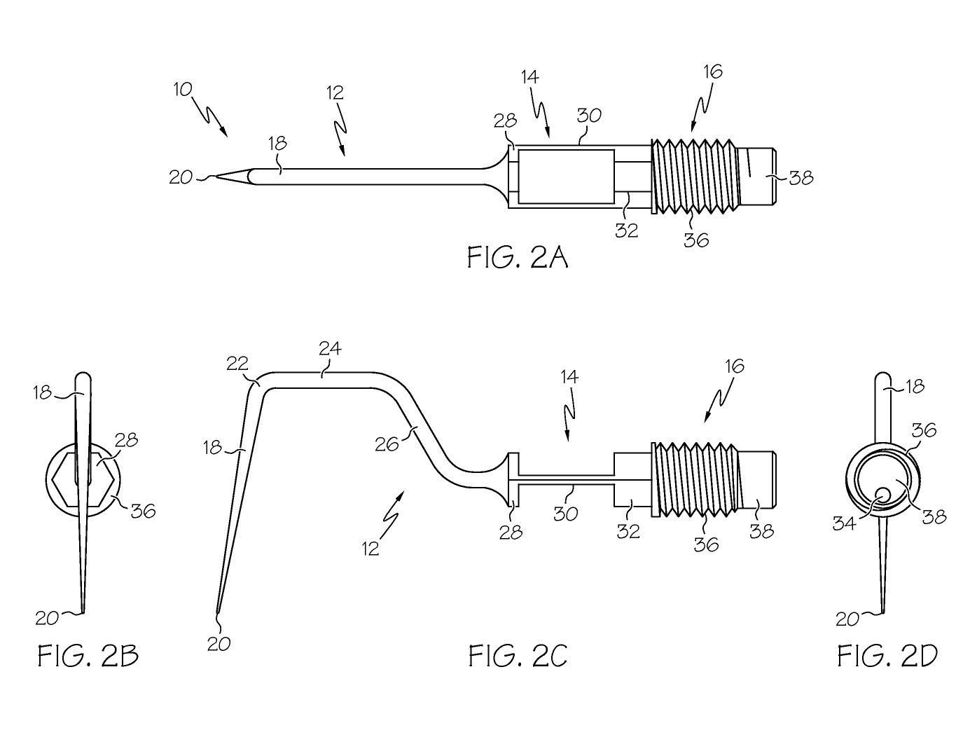

With reference now to the drawing figures, wherein like reference numbers represent corresponding parts throughout the several views, FIGS. 1 and 2A-2D show an example embodiment of the apparatus contact member 10. The depicted contact member includes a probe tip 12, a base 16 and an intermediate flexure bridge 14. The probe tip 12 is preferably constructed of a substantially rigid material that will not easily flex, such as, but not limited to, injection-molded plastic. Alternative embodiments can include contstruction of titanium, stainless steel or other metal, ceramic or polymeric materials. This probe can be disposable after use or can be re-used with sufficient sterilization. The base 16 is also preferably constructed of a substantially rigid material, such as, but not limited to, injection molded plastic. The flexure bridge 14 can be constructed of injection molded plastic, however, as described further below, in example embodiments, the bridge is capable of flexing in a predictable uni-axial direction, for example laterally and vertically and more preferably uni-axially. Optionally, the probe tip 12 can additionally be marked to indicate depth during use, and can also be marked and/or anodized to indicate intended range of application force.

The example probe tip 12 depicted in FIGS. 1 and 2 can have an overall shape resembling a typical U-shaped or C-shaped wire-form periodontal probe. This probe tip 12 has a shape that is able to reach the buccal, lingual, and interproximal gingival tissues and intrude into the sulcus surrounding a tooth and/or periodontal implant. This depicted probe tip 12 has a material-contacting member 18 with a distal end or tip 20 and a proximal end 22. The material-contacting member 18 has a generally circumferential outer surface with a generally circular cross-sectional profile, and frustoconically tapers from the wider proximal end 22 to the narrower distal end 20. The distal end 20 can have a pointed shape, a rounded shape, a flat shape, or can otherwise be shaped as desired. The proximal end 22 is coupled to or integrally molded with the front end of a forward-extending member 24, for example through injection molding. The material-contacting member 18 can be angled between about 90-110 degrees with respect to the forward-extending intermediate segment or member 24. More preferably, the material-contacting member 18 is angled about 100 degrees with respect to the forward-extending member 24. And, the forward-extending member 24 has a generally circumferential outer surface.

The end of the forward-extending member 24 opposite the material-contacting member 18 is secured to a ramp member 26. The ramp member 26 extends obliquely away from the forward-extending member 24 at an angle of inclintation of between about 110 and 130 degrees with respect to the forward-extending member. More preferably, the ramp member 26 extends at an angle of about 120 degrees with respect to the forward-extending member 24. The ramp member 26 has a generally circumferential outer surface. As shown in FIGS. 2A, 2B and 2D, the tissue-contacting member 18, the forward-extending member 24 and the ramp member 26 extend in a parallel longitudinal plane away from the flexure bridge 14 and base 16. And, preferably the distal end 20 of the tissue-contacting member 18 extends vertically below the horizontal axial level of the flexure bridge 14 as shown in FIG. 2C.

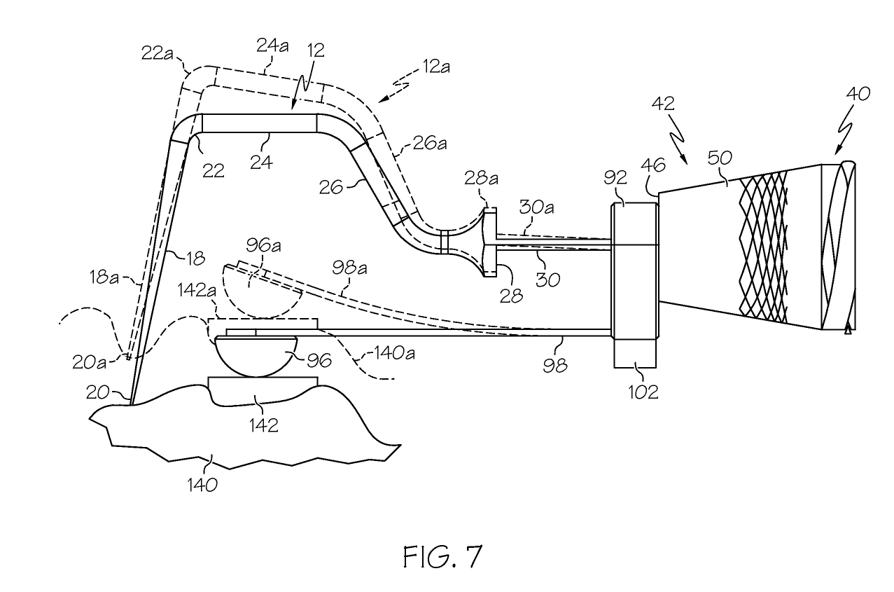

In example embodiments, the intermediate flexure bridge 14 includes a planar member or panel 30 that is separated from the probe tip 12 by a wall or flange 28. The wall 28 can be vertically perpendicular to the length of planar member 30. The wall 28 can have a variety of shapes. As shown the wall 28 can have a hexagonal shape. The planar member 30 can have a rectangular shape that flexes vertically (with respect to the orientation of FIG. 2c) at a point of connection with a coupling or gripping region 32 (shown in FIG. 7) as a result of forces applied directly upwards or downwards on the probe tip 12. The resulting physical moment causes a constant strain deformation to occur across of the planar member 30. Alternately the planar member may be adapted to flex laterally (not shown) as a result of forces applied laterally on the probe tip 12. The material and dimensions (length, width, thickness) of the planar member 30 are controlled so that the application of force at the tip 20 produces consistent strain/flexure of the planar member.

The base 16 is secured to the flexure bridge 30 at an end opposite the wall 28. As shown, the base 16 generally includes the gripping region 32, an intermediate region 36 and a rear region 38. The gripping region 32 can have a variety of shapes, for example a hexagon. Preferably, the gripping region 32 has an outer surface shape that allows a user to grip with a gripping tool (e.g., wrench) and rotate the probe 10 for tightening purposes. As shown, in the example embodiment the gripping region 32 has a shape similar to the wall 28. The intermediate region 36 can include a threaded outer surface. And, as shown, the rear region 38 is cylindrical in shape. As seen in FIG. 2D, a channel 34 extends through the length of the base 16 between the gripping region 32 and the rear region 38. As shown, the channel 34 has a cylindrical cross section.

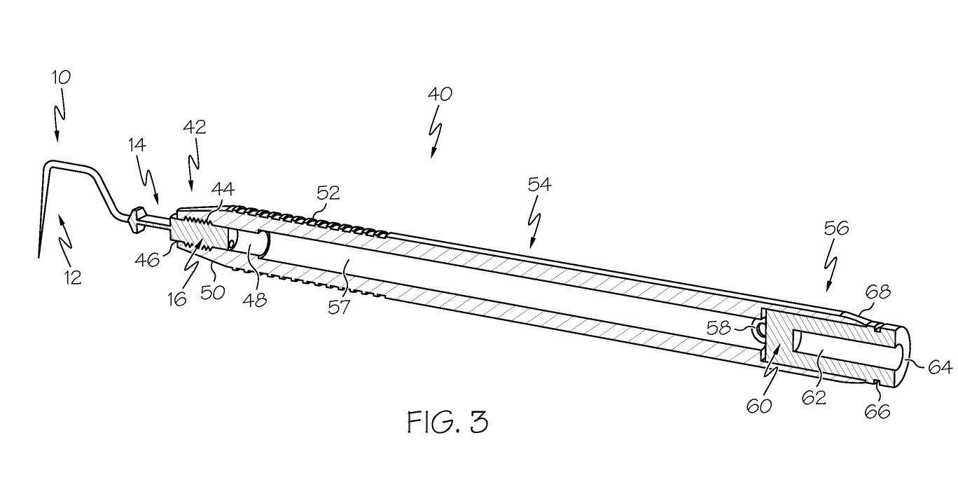

As shown in FIG. 3, the contact member 10 can be secured to a handle 40 constructed of a rigid material, for example plastic or aluminum. As depicted, the handle 40 includes a forward region 42, an intermediate region 54 and a rear region 56. The forward region 42 can have a frustoconically tapering outer surface 50 that narrows away from the intermediate region 54 toward a forward-facing surface 46. A threaded probe-receiving aperture (not shown) is located within the forward-facing surface 46. The front region also includes a probe-receiving channel 48. The probe-receiving channel 48 extends from the aperture (not shown) on the front-facing surface 46 to a second channel 57 without interruption. And, the probe-receiving channel 48 includes a threaded circumferential inner surface 44. In example embodiments, the pattern and diameter of the threaded surface 44 along the first channel corresponds with the threaded outer surface 36 of the contact member base 16. As shown, the base 16 is inserted into the aperture of the front-facing surface 46 and the threaded section 36 is secured within the threaded circumferential region 44 with a friction fit.

The second channel 57 extends the length of the intermediate region 54 of the handle 40. The second channel 57 can have an identical diameter to the first channel 48, or alternatively, the second channel 57 can have a greater or smaller diameter to the first channel 48.

As shown, the handle 40 includes an outer grip surface 52 that comprises raised concentric ridges. This grip surface 52 is located near the forward region 42 and behind the tapered surface 50.

As depicted in FIG. 3, the example embodiment comprises a rear region 56 that has a data communicator, for example an open end 68 that is shown to receive a jack 60 for transmitting electronic signals. The jack 60 includes an outwardly-facing end 64. An electronic communication portal 62 is located within the jack 60 and provides an opening on the outwardly-facing end 64 and in internal channel 62. The internal channel 62 is adapted to receive an electronic communication cable, for example a 3.5 mm AV cable, a USB cable, a wireless transmitter for communication with a remote device, for example a display or receiver, or any other communication vehicle. The jack 60 is insulated with a hard insulating material so that it can be self-sealed and press fit into the open end 68 of the handle 40. Preferably, the jack 60 can be removed from the handle 40 for cleaning. The jack 60 can also include an annular notch 66 that allows a user to grip the jack and remove it from the handle 40. And, the jack 60 includes a wire harness or anchor 58 to secure wires that extend from, and communicate with, the contact member 10. The wire anchor 58 can transmit electronic signals and communicate with between 1 and 4 wires, or more than 4, and more preferably 2 to 4 that extend from the probe. Alternatively, the data communicator can include an analog display (not shown) that is mechanically manipulated by an applied force on the probe.

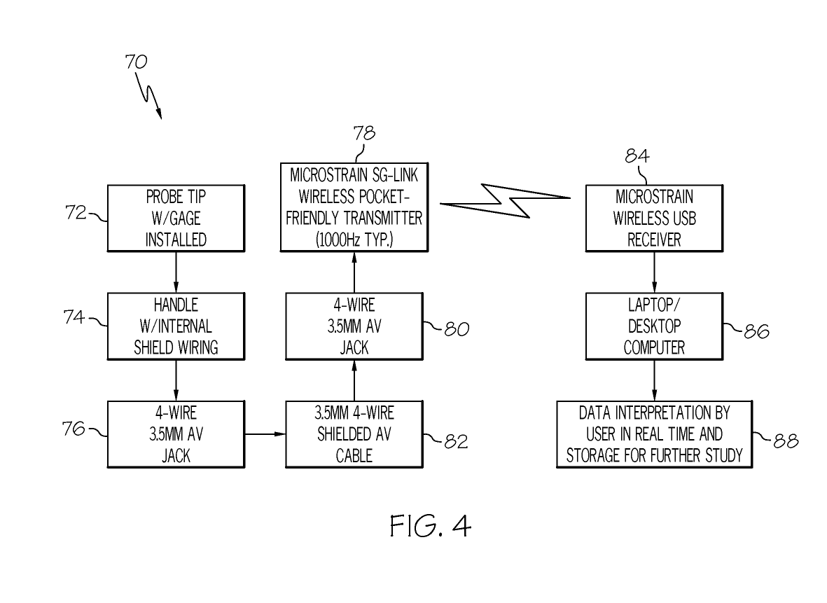

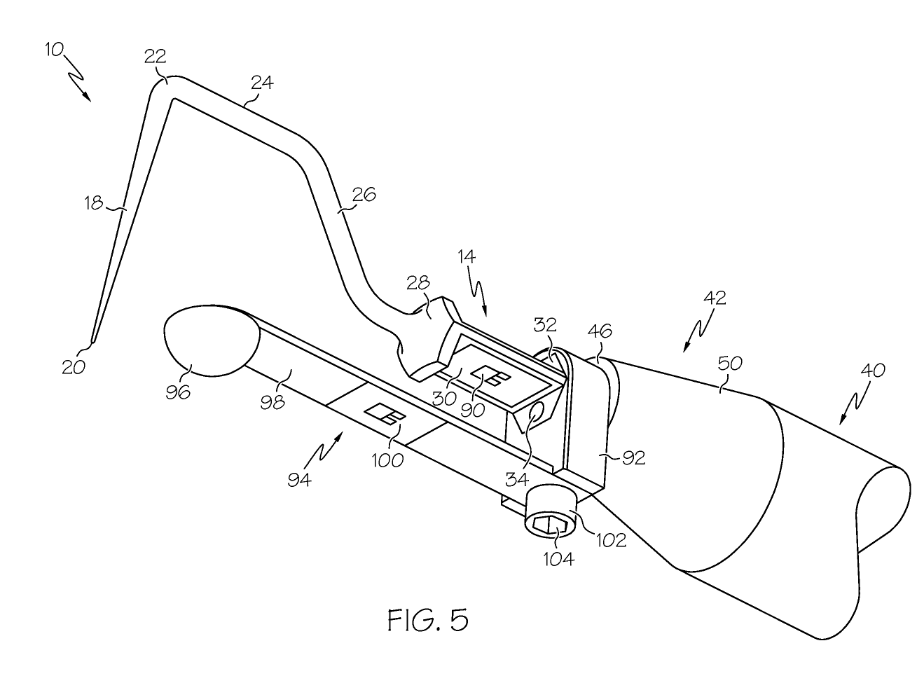

A sensor measures the strain values imparted on the flexure bridge 30 resulting from the application of force on the tip 20. The sensor includes a dynamic electrical component adapted to flex with respect to the bridge 30. This dynamic electrical component can produce a variable electrical output and can be a strain gauge, a piezoelectric conductor and a transducer. A typical strain gage can measure strain in the linear direction either vertically or laterally. The sensor is preferably secured to the upper or lower surface of the planar member 30 of the flexure bridge 14. As shown in FIG. 5, the sensor 90 can be secured to the bottom side of the flexure bridge 30 because during probing, the bottom surface of the flexure bridge 30 is the side that is under tension. Shielded, insulated wiring (not shown) is fed from the sensor 90 through the channel 34 in the base 16 and then routed inside of the handle 40 to the jack 60 (schematically represented in FIG. 4). Sensor wiring typically includes between 2 and 4 wires. The surface of the flexure bridge 30 on which the sensor 90 is secured is sealed with a flexible acrylic to prevent entry of fluid. Alternatively, this sensor 90 can be secured with a fastener or an adhesive. The opening of the channel 34 is also sealed with a flexible acrylic once the wires (not shown) are inserted through the opening.

A method of using an apparatus for material characterization according to an example embodiment of the present invention is shown in FIG. 4. The contact member with a secured sensor is installed at step 72 within the handle. The shield wiring is inserted at step 74 and secured within a handle and run along the handle length to connect with a data communicator at steps 76, 82, for example an AV jack, that can include between 2 and 4, or more, 3.5 mm wires. The data communicator can communicate electronically with a wireless transmitter at step 78, for example a MICROSTRAIN SG_LINK pocket-friendly transmitter. The transmitter wirelessly transmits a signal to a receiver at step 84, for example a MICROSTRAIN USB receiver that communicates with a computer at step 86. The signal transmits data and the computer interprets the data in real time at step 88 to be stored and/or for further study.

The described can communicate via wired or wireless connection with a remote or onboard device capable of resolving electrical differences in resistance of the described strain gage and returning real strain values in real time. A typical value of electronic communication is 1000 Hz. The geometry of the described probe, in conjunction with measured strain values and material properties, is used to calculate the approximate force applied. A graph of the strain with respect to time is transformed into a plot of deformation with respect to time, and then integrated. The resulting unit for this measurement is Newton-seconds (i.e., an energetic measurement describing change in momentum). The real-time graph of strain gives an indication of tissue rupture and force ramping (i.e., evidence of contact with a hard surface, for example a bone), while the integrated result quantifies the energetic resources required to reach the hard surface. This quantification can be used to characterize the quality with which soft tissue is attached, in-vivo, to important surrounding structures. An example remote device is a computer that can record data, process or convert signals received to values relevant to the practitioner, display data graphically, compare measured values with target ranges or threshold values.

FIG. 5 shows the contact member 10 in use with a displacement sensor instrument 94 that determines displacement relative to the position of a presumably fixed geometry body, for example an implant, tooth, and/or prosthesis. The example displacement instrument 94 includes an elongated bridge 98 with a bulbous protrusion or contact 96 at a distal end. A proximal end of the bridge 98 is secured with respect to a mounting foundation 92 with a nut 102 that can be tightened through an aperture 104. According to example embodiments, the displacement instrument 94 includes a sensor 100 that measures linear strain similarly to the sensor 90 of the contact member 10. The example sensor 90 is a sensor with resistance that varies with applied force. The example sensor 90 converts force, pressure, tension, weight, etc., into a change in electrical resistance, which can then be measured. An example sensor is constructed of bonded foil, for example a strain gauge.

The displacement instrument 94 operates on the same wiring and physical principles of strain generation as the probe tip 12, but pivots off a stable feature on the top of the fixed geometry structure, which has proximal tissue being characterized. While the contact member 10 can sense depth to the point of first tissue contact by an algorithm that does not commence recording force data until a specific threshold has been met (i.e., that of a nominal contact force between probe tip and tissue), the displacement instrument 94 produces data that can include overall displacement and distinguish regions of displacement that involve tissue contact and those that do not. Monitoring of strain and displacement rates can notify the user when a hard surface has been contacted through a typical signal communication device (e.g., transmitter), and therefore, when all soft tissue has been displaced. The data, all recorded in real time, is sufficient for determining the basic viscoelastic response of the tissue in question. The algorithm used for the displacement instrument 94 optionally also accounts (to a reasonable degree) to the tremor susceptibility of a user's hand by deleting corresponding force/time data that is not in a uni-axial downward progression with respect to displacement.

Preferably, this displacement instrument 94 is used in-vivo and in a clinical setting, but can alternatively be operable as attached to a stable linear actuator through a periodontal force probe holster 92, and ready-set to quickly interface testing equipment such as a linear actuator manufactured by MTS Systems, Corporation.

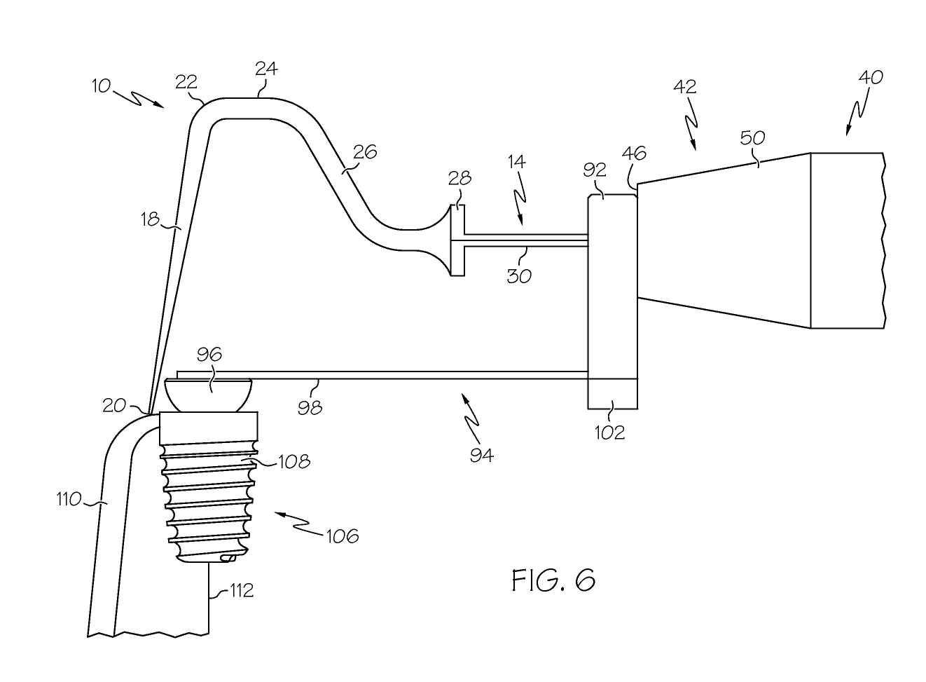

FIG. 6 shows an example of how the at-rest relaxed probe 10 and displacement instrument 96 interface a fixed geometry 106 and biologic tissue 110 of a human or animal subject. In FIG. 6, the tissue 110 has been sectioned to reveal the relative position of the fixed geometry to the displacement instrument 94 and the contact member 10. The bridge 98 of the displacement instrument 94 can flex compared with the bridge 30 of the contact member 10, and the bridge 98 of the displacement instrument 94 can be designed to interface with or be adjusted to accomodate a variety of rigid structure heights. A sensor communicates with respect to the bridge 98. Preferably, the sensor (shown in FIG. 5 as 100) can be secured to the under surface of the bridge 98. The contact member 10 is designed to contact and characterize material, for example the lingual and interproximal tissues. A further attachment (not shown) extending beyond the probe tip 20 can take mechanical readings for buccal tissue.

The contact member 10 is shown in FIG. 7 in a relaxed state (solid lines, as described with respect to FIG. 6) and in a deflected state (shown in dashed lines), both with respect to a fixed geometry (e.g., tooth) 142, 142a and gum tissue 140, 140a. As shown in the deflected state, the distal end 20a of the tissue-contacting member 18a is exerting a downward force into a pocket/sulcus of gum tissue 140a near the fixed geometry 142a. This downward force is applied by a user through the tool handle 40. Equally, this downward or lateral force is met with resistance from the tissue 140a and correspondingly causes the flexure bridge 30a to flex/bend upwardly or laterally. The resistive force of the tissue 140a causes a corresponding movement of the stopper 28a, the ramp 26a, the forward-extending member 24a, and the proximate 22a and distal 20a ends with respect to the plane of the handle 40. The strain on the flexure bridge 30 caused by this displacement is measured by the sensor 90 (shown in FIG. 5) described above. As further shown, the upward force caused by the fixed geometry 142a forces the displacement instrument 98a to correspondingly flex, while maintaining contact between the bulbous end 96a with the fixed geometry 142a. The flexing movement of the contact member 10 and the displacement attachment 98 is a strained similarly to the displacement of the flexure bridge 30. Therefore, upon application of a force, the strain measured by the sensor (not shown) secured to the displacement instrument 98a against the fixed geometry 142a can be compared with the strain caused in the probe 12a against the tissue 140a. Typically, gum tissue in poor health will create less strain on the probe 12a than healthy gum tissue. In other words, healthy gum tissue will provide more resistance against the force applied by the tissue-contacting member 18a.

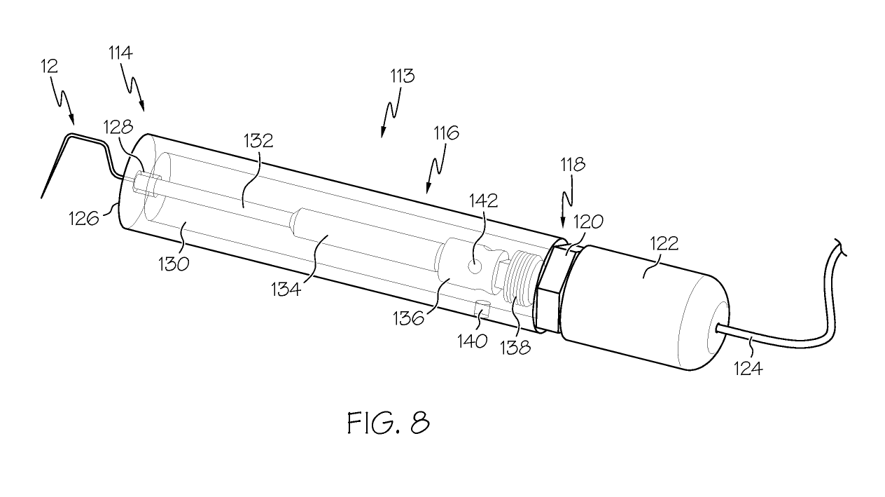

FIG. 8 shows a further example embodiment in which the probe 12 is secured (without displacement sensitivity) to a pressure transducer 113, for example a load cell. A typical load cell is used to convert a mechanical force into electrical signal. The depicted load cell 113 includes a front region 114, an intermediate region 116 and a rear region 118. An example load cell that is compatible with a probe of the present invention is the Futek Model LPM200. The probe 12 is secured within a first channel 128 extending inwardly from a forward-facing surface 126 in the front region 114. The first channel 128 extends from the forward-facing surface 126 until abutting with a second channel 130 that extends throughout the intermediate region 116 and the rear region 118. The probe base (not shown) is secured within the end of a metal member 132. The metal member 132 is secured to a bracket 136 with a fulcrum and/or pivot pin 142 that is inserted into a pivot notch 140 proximal to the load cell surface, and placed in interference with the load cell surface. A threaded portion 138 of the load cell 113 in conjunction with a tightening member 120 fastens the load cell to the intermediate region 116. The load cell 113 is secured to a power source 122 and communication source 124 through the tightening member 120. The communication source 124 can be a wireless transmitter or a wire-connected AV jack or USB jack. Upon typical probing with the probe tip 12, a moment arm is created by the upward rotation of the probe 12, and this information is transmitted directly to the load cell, and an algorithm can be used to determine the actual force applied by the tip to gum tissue.

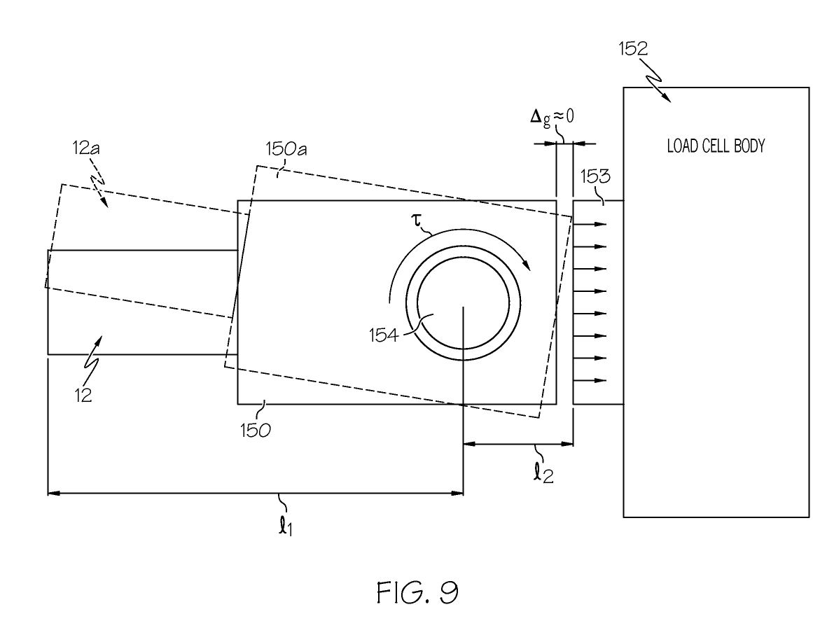

FIG. 9 is a diagram representing the interaction of the probe tip 12 and probe tip base 150 with a load cell body 152. The example load cell body 152 includes a pressure transducer sensor 153 secured through securing means (e.g., adhesive, fastener, tongue and groove) a defined distance away from the rear surface of the load cell base 150. The probe shaft base 150 is secured on a pivot pin 154 so that the probe can rotate upwardly and downwardly with respect to the pressure transducer sensor 153. As shown, as a downward force is applied on gum tissue by the probe tip 12, the tip 12a rotates upwardly with resistive force applied by gum tissue (F_{applied}). As the tip 12 rotates, the probe base 150 correspondingly rotates and the upwardly-rotated base 105a contacts the pressure transducer 153 with a particular force (F_{recorded}) recorded by the load cell. In a preferred embodiment there is substantially no gap between the base 150 and the pressure transducer 153. Assuming there is substantially no gap, the rotational (τ) movement of the base 150 around the pin 154 is equivalent to the force applied (F_{applied}) to the probe tip multiplied by the length (l₁) of the probe shaft. This rotational movement (τ) is also equivalent to the force recorded (F_{recorded}) by the pressure transducer 153 multiplied by the distance (l₂) between the center of the pin 154 and the front-facing surface of the transducer 153. Thus, the relationship is defined as follows:

τ=movement=F_{applied}×l₁=F_{recorded}×l₂

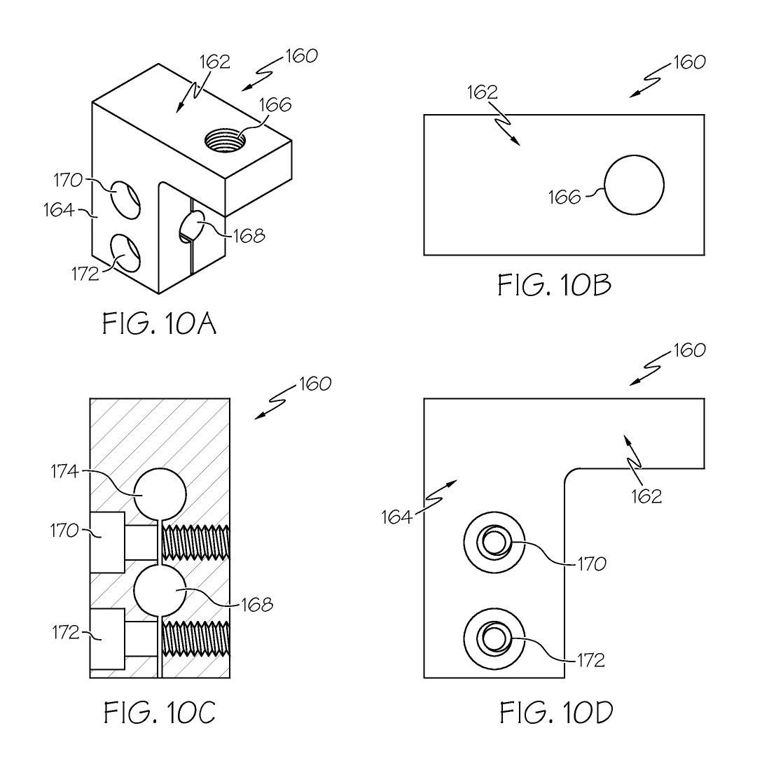

FIGS. 10A-10D show an example probe mount 160 that can be used to mount the probe and the displacement attachment (described above) to a typical linear actuator, for example a linear actuator manufactured by MTS Systems, Corporation. This example probe 160 includes a base 164 and an extension 162. As shown, the base can have two horizontally-extending apertures 170, 172 that can receive and secure fasteners. The base 164 can additionally have two longitudinally-extending apertures 168,174. And, the extension 162 can have a vertically-extending aperture 166. Preferably, the extension 162 inserts into the aperture in the front-facing side 46 of the handle 40 (shown in FIG. 3). The probe base 16 (shown in FIGS. 1 and 2) can secure within the aperture 170 of the mount 160. And the displacement attachment 94 can secure within aperture 172.

While the invention has been described with reference to preferred and example embodiments, it will be understood by those skilled in the art that a variety of modifications, additions and deletions are within the scope of the invention, as defined by the following claims.

What is claimed is:

1. An apparatus for material characterization comprising:
a contact member for applying a force to a defined section of material;
a sensor to record the force applied onto the material by the contact member; and
a data communicator to communicate the recorded force.

2. The apparatus of claim 1, wherein the data communicator comprises an analog display manipulated by the applied force of the contact member.

3. The apparatus of claim 1, wherein the data communicator is in electronic communication with the sensor.

4. The apparatus of claim 1, wherein the data communicator comprises a transmission interface to transmit recorded data to a data processor.

5. The apparatus of claim 4, wherein the transmission interface transmits data through wireless communication.

6. The apparatus of claim 4, wherein the transmission interface detachably connects to a data recorder.

7. The apparatus of claim 1, wherein the contact member comprises a probe.

8. The apparatus of claim 7, wherein the probe is adapted to characterize the tissue in the region surrounding a periodontal element.

9. The apparatus of claim 7, wherein the probe comprises a wire-form periodontal probe.

10. The apparatus of claim 7, wherein the probe is removably secured with respect to a handle.

11. The apparatus of claim 1, wherein the contact member comprises a region having predictable flexibility.

12. The apparatus of claim 11, wherein the predictable flexibility comprises flexibility in a uni-axial direction.

13. The apparatus of claim 11, wherein the predictable flexibility comprises flexibility in a lateral direction.

14. The apparatus of claim 11, wherein the sensor comprises a dynamic electrical component adapted to flex in correspondence with a surface of the region having predictable flexibility.

15. The apparatus of claim 14, wherein the dynamic electrical component is secured with respect to the surface of the region having predictable flexibility.

16. The apparatus of claim 14, wherein a flex of the dynamic electrical component produces a variable voltage output.

17. The apparatus of claim 14, wherein the dynamic electrical component comprises one of a group comprising a strain gauge, a piezoelectric conductor and a transducer.

18. The apparatus of claim 1, further comprising a displacement instrument secured with respect to the contact member, wherein the displacement member recognizes the physical displacement of the contact member with respect to a fixed geometry secured within the defined section of material to be characterized.

19. The apparatus of claim 18 further comprising a means for indicating the instantaneous value of recorded force and physical displacement.

20. The apparatus of claim 18, wherein the displacement instrument comprises a flexible member influenced by a resistive force applied by the fixed geometry.

21. The apparatus of claim 20, wherein the resistive force is mechanical.

22. The apparatus of claim 19, wherein the displacement instrument comprises a sensor to communicate the resistive force applied by the fixed geometry.

23. The apparatus of claim 22, wherein the sensor comprises a dynamic electrical component adapted to flex in correspondence with the displacement member flexible member.

24. The apparatus of claim 1, wherein the contact member comprises a lever arm.

25. The apparatus of claim 24, wherein the lever arm is pivotally secured on a fulcrum.

26. The apparatus of claim 25, wherein the fulcrum comprises a pivot pin.

27. The apparatus of claim 24, wherein the sensor comprises a pressure transducer that records force data upon being contacted by the pivotally secured lever arm.

28. The apparatus of claim 1, wherein the contact member is displosably in communication with respect to a data communicator.

29. An apparatus for material characterization comprising:
a predictably flexible member for applying a force to a defined section of material;
a sensor secured with respect to the flexible member, wherein the sensor records strain data generated by the flexible member upon application of the predictably flexible force to the material; and
a communication interface in communication with the sensor, wherein the communication interface relays the strain data.

30. The apparatus of claim 29, wherein the sensor is a strain gage.

31. An apparatus for material characterization comprising:
a force-application member that physically reacts to a resistive force generated by the material upon which a force is applied;
a sensor that measures the physical reaction of the force-application member in the form of data; and
a data communicator that communicates the data recorded by the sensor.

32. The apparatus of claim 31, wherein the sensor is a load cell.

33. A system for characterizing material comprising:
a contact member for applying a force to a defined section of material;
a sensor to record the force applied onto the material by the contact member;
a data communicator to communicate the recorded force; and
a data receiver to receive the communicated recorded force.

34. The system of claim 33, further comprising a display that displays the recorded force received by the data receiver.

35. A method for characterizing material comprising the steps of:
applying a force to a material with a contact member comprising predictable flexibility;
recording a material resistive force with a sensor in flexible communication with respect to the contact member; and
communicating the recorded resistive force through a data communicator.

36. The method of claim 35, wherein the material upon which the force is applied comprises biologic material.

37. The method of claim 36, wherein the material resistive force comprises the compliance of the biologic material.

38. The method of claim 35, wherein the material upon which the force is applied comprises gingival tissue.

38. The method of claim 38, further comprising relating the resistive force to a physical condition of the gingival tissue.

39. An apparatus for material characterization comprising:
a contact member for applying a force to a defined section of material;
a sensor to record the force applied onto the material by the contact member; and
a displacement instrument secured with respect to the contact member, wherein the displacement member recognizes the physical displacement of the contact member with respect to a fixed geometry secured within the defined section of material to be characterized.
